(19) United States
(12) Patent Application Publication
(10) Pub. No.: US 2024/0299153 A1
(43) Pub. Date: Sep. 12, 2024

(54) IMPLANT

(71) Applicant: OLYMPUS WINTER & IBE GMBH, Hamburg (DE)

(72) Inventors: Kevin Alexander SCHULZ, Wilstedt (DE); Ido KILEMNIK, Henial (IL)

(73) Assignee: OLYMPUS WINTER & IBE GMBH, Hamburg (DE)

(21) Appl. No.: 18/414,787

(22) Filed: Jan. 17, 2024

Related U.S. Application Data
(60) Provisional application No. 63/450,265, filed on Mar. 6, 2023.

(51) Int. Cl. A61F 2/04

(52) U.S. Cl. CPC A61F 2/04; A61F 2002/047; A61F 2210/0004; A61F 2220/0008; A61F 2220/0091; A61F 2230/0023; A61F 2250/0036; A61F 2250/0068

(57) ABSTRACT

An implant that can be used for the treatment of the urinary tract in a manner which is more efficient and gentler on the body. This is achieved by virtue of the fact that an implant for expanding a urethra of a person by applying a local ischemic pressure to the tissue of the urethra has a pressure means structure. This pressure means structure has at least two pressure means, wherein the implant can be introduced into the urethra with a distal end in front. In this case, each of the pressure means has a spreading element and two bracing elements, namely a proximal and a distal bracing element, and wherein the bracing elements are each articulated movably at a first end to the spreading element and movably at a second end to a central bracing means.

The invention relates to an implant in accordance with the preamble of claim 1.

Various methods and techniques are known for the treatment of the urinary tract, in particular benign prostatic enlargement (benign prostatic hyperplasia, BPH). In a minimally invasive treatment of BPH symptoms which is particularly gentle on the body, a removable implant is temporarily placed in the urethra or in the prostatic portion of the urethra of the patient. Such an implant is a wire structure made of a shape memory alloy, such as, for example, Nitinol. In a folded state, the wire structure is pushed into the correct position by a catheter in order to unfold there into its predetermined basic structure. This structure, which can be formed from three or four wires, is a basket structure. This basket structure widens the urethra. Owing to the expansion of the wire structure against the tissue of the urethra, the stricture tissue of the urethra is denatured over the course of a few days. This denaturation of the tissue takes place on account of the ischemic pressure of the individual wires on the cells of the tissue, leading to reduced or completely absent blood flow. As a result, the lack of blood supply leads to a lack of oxygen in the cells and ultimately to death of the cells. Within a few days, the tissue can be reduced to such an extent that the urinary flow almost normalizes. After completion of this treatment, the implant can be recovered from the urethra by means of a catheter.

It must be regarded as particularly disadvantageous that the implant has to be explanted after the treatment of the prostate. To do this, the patient must visit a doctor again and undergo further treatment. This treatment involves further effort and is at least not completely risk-free from a medical point of view. Removal of the implant can lead to further traumatization of the treated area.

Proceeding therefrom, the underlying object of the invention is that of providing an implant that can be used for the treatment of the urinary tract in a manner which is more efficient and gentler on the body.

One way of achieving this object is described by the features of claim 1. Accordingly, it is envisaged that an implant, in particular a removable implant, for expanding a urethra of a person by applying a local ischemic pressure to the tissue of the urethra has a pressure means structure. This pressure means structure has at least two pressure means, wherein the implant can be introduced into the urethra with a distal end in front. In this case, each of the pressure means has a spreading element and two bracing elements, namely a proximal and a distal bracing element, and wherein the bracing elements are each articulated movably at a first end to the spreading element and movably at a second end to a central bracing means. By virtue of this structure of the aforementioned components of the implant, which are movably connected to one another, the implant can be introduced into the urethra according to the principle of a folded umbrella and opened there in order to exert the necessary pressure on the tissue to be treated. By overstretching the implant or the individual components, the diameter of the implant can be reduced again, as with an umbrella, in order in this way to move the implant in the urethra or remove it therefrom, as necessary. This makes the treatment of the patient particularly efficient and gentle on the body.

Provision is preferably made for the two ends of the bracing elements each to have a joint, by means of which they are connected movably, preferably in a latching manner, to the respective spreading element and to the central bracing means. These joints ensure that the respective pair of interconnected components moves in a common or defined plane and thus that the implant can be opened exactly as intended in order to carry out the treatment.

It is preferably conceivable for the pressure means structure to be made up of three pressure means, wherein the three pairs of bracing elements are movably connected by their second ends to the common central bracing means and to a respective spreading element. The pressure means thus extend around the common bracing means and exert the required pressure on the surrounding tissue. One specific exemplary embodiment of the invention envisages that the three spreading elements of the three pressure means are arranged at an angular spacing of 120° with respect to one another around the central bracing means. This symmetrical arrangement of the spreading elements around the bracing means makes it possible to ensure that the pressure exerted on the tissue is symmetrical, thereby enabling particularly efficient treatment.

In the above-described exemplary embodiment of the invention, provision can be made for the spreading elements of a plurality of pressure means to be aligned parallel to one another. This parallel alignment of the, in particular rod-type, spreading elements also results in particularly uniform application of pressure to the surrounding tissue.

Provision can furthermore be made, according to the invention, for the pressure means to be brought into a folded state, wherein the bracing elements and the spreading elements can be moved in the direction of the bracing means, thereby minimizing the distance between the bracing elements, the spreading elements and the bracing means. In this folded state, the implant can be brought into the area of the urethra to be treated in a particularly simple and advantageous manner. In this state of minimum diameter, the implant has a particularly gentle effect on the patient's body.

Another advantageous exemplary embodiment of the invention can provide for the pressure means to be brought into an opened state, wherein the bracing elements and the spreading elements can be moved away from the bracing means, thereby maximizing the distance between the spreading elements and the bracing means, and the spreading elements thus open up the urethra. The implant is accordingly opened within the urethra according to the umbrella principle, wherein the person administering the treatment performs the opening process. This opening of the implant can be done precisely to the extent necessary for the efficient treatment of the urethra. Thus, exactly the pressure necessary to successfully treat the tissue can be applied to the tissue. The application of too high or too low a pressure can thereby be avoided.

In particular, the invention can provide for the bracing means to have a distal and a proximal end, wherein it can be pushed into the urethra with the distal end in front, and wherein the pressure means structure with the pressure means can be opened by the action of force on the proximal end in the proximal direction. This application of force can take place, for example, via a thread or a rod, which is connected to the bracing means. Alternatively, it is also conceivable for the bracing means to be pulled in the proximal direction by forceps or the like. By pulling the bracing means in the proximal direction, the pressure means move outward perpendicularly to the pulling direction, wherein the spreading elements and the bracing means rotate about the joints. The further the bracing means is pulled in the proximal direction, the further the pressure means open, thereby maximizing the diameter of the implant. When the bracing means encloses a right angle with the bracing elements, the implant has reached its largest possible diameter. Whether this is necessary for the treatment of the urethra must or can be decided on a patient-specific basis.

Another exemplary embodiment of the invention can provide for the distal bracing elements each to have an extension which projects beyond the first end, wherein, in the opened state, the pressure means project with the extensions in a bladder neck and can thus be fixed in the urethra. When positioning the implant within the urethra, the implant is introduced into the bladder neck. Opening the pressure means structure causes the distal ends of the bracing elements to settle on the bladder neck and act as a kind of anchor for the implant within the urethra. As the implant is opened further, the distal ends move outward. In order to simplify this movement of the distal ends of the bracing elements along the bladder neck, one exemplary embodiment can provide for the extensions to have curvatures, which are preferably of arcuate design. By means of this arc, it is possible to ensure that the bracing elements slide over the tissue and do not have a traumatizing effect.

It is preferably conceivable for the joints between the spreading elements and the bracing elements and between the bracing elements and the bracing means to be rotatable only over a limited angular range (angle limitation). As a result of this limitation of the available angular range, the implant can, at least initially, be brought at most into a state with a predetermined opening angle or diameter of the pressure means structure. For the placement of the implant, it is therefore not possible to open the structure beyond the state of the maximum diameter. Incorrect operation of the implant can thus be avoided.

It is furthermore conceivable for the joints to have a latch between the spreading elements and the bracing elements and between the bracing elements and the bracing means, thus enabling them to be brought into a selectable relative angular position. This fixing or locking can be used to fix the selected diameter of the implant. The latching of the joints enables the structure to be locked synchronously in an optimized angular position. The implant remains in this fixed position for the entire treatment of the tissue.

Another possible exemplary embodiment of the invention can provide for the angular limitation of the joints to be lifted by the application of force to the bracing means in the proximal direction, and for the pressure means to be brought back into a folded state via the opened state in order to pull the implant out of the urethra. This folded state corresponds precisely to the folded initial state of the implant, with the joints or the components arranged thereon being rotated by 180°. The exertion of force on the bracing means can take place via a thread, a bar or a rod. The person administering the treatment usually notices if the joints break during the exertion of the force. A sudden relaxation of the counterforce alerts the person administering the treatment to the fact that the joints have gone beyond the angle limitation and that the implant can now be pulled out of the urethra. One possible exemplary embodiment can provide for the latching means of the joints to dissolve over time, so that no action of force is necessary to bring the implant into a folded state.

The invention furthermore preferably provides for the spreading elements to be of hollow design for the metered reception of a substance, preferably a medicament. This substance can be dispensed in a metered manner by the spreading elements and absorbed by the body. The substance can be dispensed through openings or the like in the spreading elements. It is likewise conceivable for the spreading elements to dissolve over time and for the substance to be dispensed as a result.

In particular, it is possible, for this specific exemplary embodiment of the invention, to provide for the spreading elements to have a polygonal, a triangular or drop-like cross section, wherein the spreading elements are oriented in such a way that they are aligned with an acute angle of the cross section in the direction of the tissue of the urethra to be treated. This shaping of the spreading elements and their orientation make it possible to exert a high pressure on the surrounding tissue. This increased exertion of pressure is particularly advantageous for treatment since the tissue degrades particularly quickly as a result.

It is also conceivable for the spreading elements and/or the bracing means and/or the bracing elements and/or the joints to be produced from a biodegradable material, preferably a plastic. Owing to the fact that the aforementioned components are biodegradable, they dissolve automatically. A renewed intervention to remove the implant is thereby rendered superfluous. The slow dissolution of the structure also reduces the action of force on the tissue. Moreover, the mechanical stress of the opened structure slowly decreases, allowing the implant to move independently in various directions, where it can ultimately dissolve completely.

Finally, it is conceivable according to the invention that the spreading elements, the bracing means and the bracing elements have different thicknesses or wall thicknesses, as a result of which the spreading elements, the bracing means and the bracing elements degrade at different rates. Owing to the fact that the aforementioned components degrade at different rates, it is possible to predetermine the further location and the further state of the implant within the urethra. It is conceivable that rapid degradation of the bracing elements will cause the entire implant to move into the bladder neck. Appropriate selection of the wall thickness of the spreading elements enables the delivery of a medication to be controlled in a specifically intended way. Similarly, appropriate selection of the strength of the bracing means enables the pressure-exerting structure to be kept stable for as long as possible.

It is furthermore conceivable that the pressure means structure described here has further elements for fixing within the urethra, in particular a spring, a tongue or the like, wherein these elements are designed in such a way that they degrade first.

A preferred exemplary embodiment of the present invention is explained in greater detail below with reference to the drawing. In this drawing:

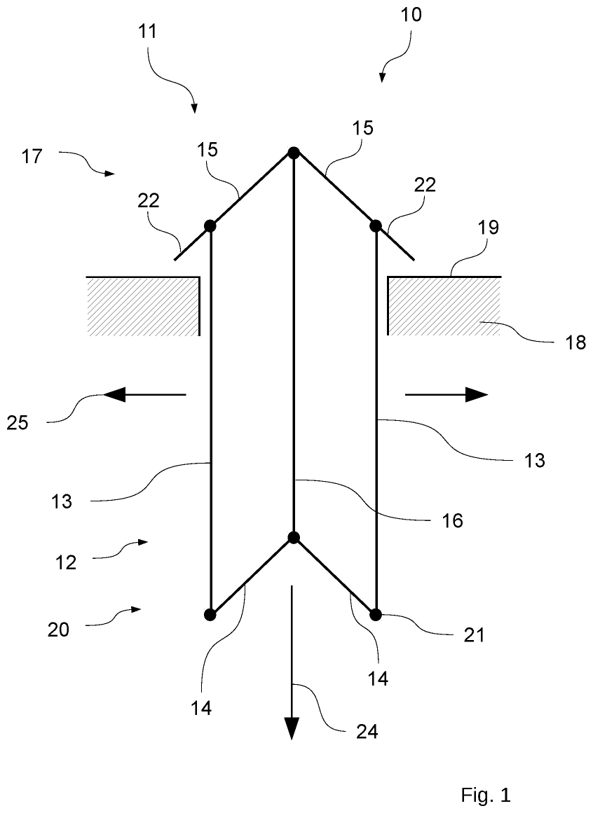

FIG. 1 shows a schematic view of an implant during positioning within a urethra,

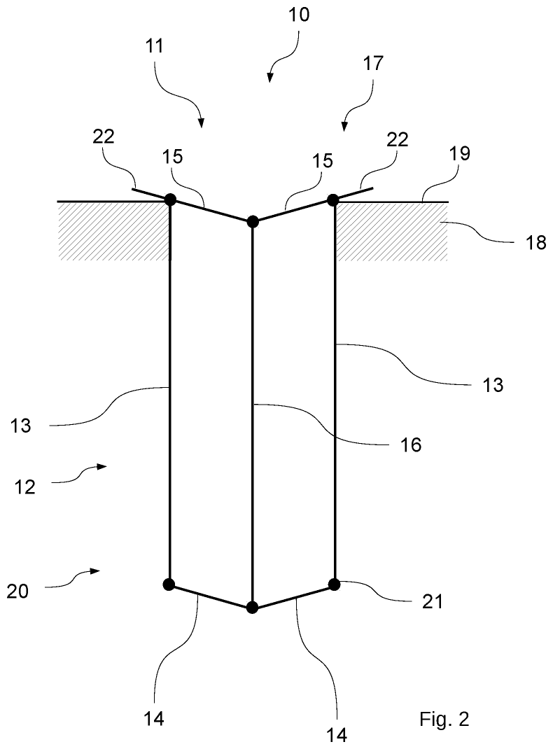

FIG. 2 shows a schematic illustration of the positioned implant,

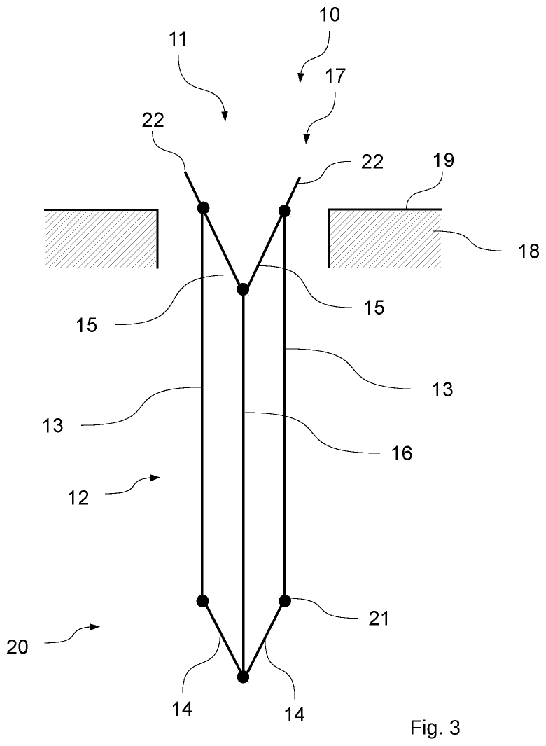

FIG. 3 shows a schematic illustration of a folded implant, and

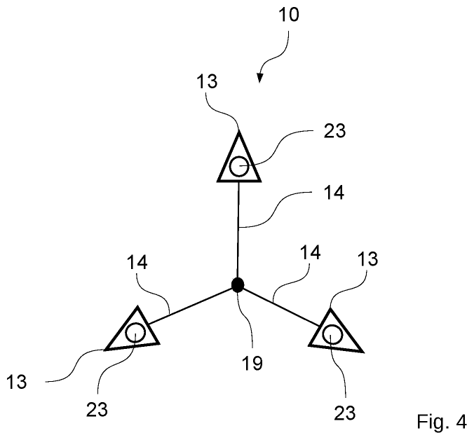

FIG. 4 shows an illustration of a cross section of an implant.

In the figures, one possible exemplary embodiment of an implant 10 according to the invention is illustrated in a highly schematic way. In a folded state, this implant 10 is pushed into a urethra 18 with a distal end 17 in front. In FIG. 1, the urethra 18 is shown as a hatched area in the plane of the page. Directly adjoining the urethra 18 is the bladder neck 19. As already explained, the implant 10 shown here serves to expand the urethra 18 and to influence the body tissue by means of the pressure applied thereto. It is of fundamental importance here that the implant 10 is introduced into the urethra 18 in a manner which is particularly gentle on the body and removed from the urethra 18 again in a manner which is likewise gentle on the body.

The exemplary embodiment of the implant 10 shown here is formed by a pressure means structure 11, which has three pressure means 12. These pressure means 12 are formed by a plurality of rods. In this case, three spreading elements 13 extend parallel to a central bracing means 16. These rod-like spreading elements 13 are each connected to the bracing means 16 via two bracing elements 14, 15. In this case, the central bracing means 16 is connected to the spreading elements 13 at the distal end 17 by distal bracing elements 15. Accordingly, at a proximal end 20 of the implant 10, the bracing means 16 is connected to the spreading elements 13 by means of proximal bracing elements 14. The pressure means 12 thus form a parallelogram.

These three parallelograms or pressure means 12 forming the implant 10 are designed to be movable. For this purpose, a joint 21 is assigned to each of the spreading elements 13 at the points of contact with the bracing elements 14, 15 and the bracing means 16. In the figures, these joints 21 are represented by solid circles. For the sake of clarity, only one of these solid circles is provided with a reference number.

The joints 21 are designed in such a way that the aforementioned components can be moved over a limited angular range. Thus, the implant 10 can be opened from a folded state and moved into an opened state. While the implant 10 has a minimum diameter in the folded state, the implant 10 has a maximum diameter in the opened state. Between these states, the implant 10 can be adjusted continuously or in stages. It is conceivable for the joints 21 to have a latch, by means of which the pressure means structure 11 is locked in various opened states. When the implant 10 is moved beyond the maximum opened state by an increased application of force, the joints 21 break and the bracing elements 14, 15 flip over, with the result that the distance between the spreading elements 13 and the bracing means 16 decreases and the diameter of the implant 10 is minimized.

For anchoring or locking the implant 10 within the urethra 18 or the bladder neck 19, the distal bracing elements 15 can each have an extension 22. As illustrated in FIG. 1, this extension 22 can be of straight or arcuate design in order to be able to slide better along the tissue.

One particular exemplary embodiment of the invention can provide for at least almost all the components of the implant 10 to be produced from a biodegradable material. The material can be a plastic or a structure made of magnesium. The diameter of the structures is less than 1 mm and is preferably braided from a plurality of individual wires or threads, wherein the individual wires or threads have a diameter of 1/10 mm. The biodegradability results in the individual components degrading in the body over time, making recovery of the implant 10 superfluous. The patient therefore does not have to go to the doctor again in order to undergo a further procedure. In this exemplary embodiment, it is also conceivable for the various components to have different thicknesses, ensuring that the various components degrade at different rates. By means of these different lifetimes, it is possible to control exactly how the implant 10 degrades. At the same time, it is conceivable, in particular, for the components to be such that the implant 10 migrates in the direction of the bladder neck 19 and completely degrades within the bladder.

FIG. 4 shows a highly schematic cross section of the implant 10 with three spreading elements 13, which are connected to the bracing means 16 via a proximal bracing element 14. According to the exemplary embodiment illustrated in FIG. 4, the cross section of the spreading elements 13 is triangular. However, it is also conceivable for the cross section to be drop-shaped or to have another shape with an acute angle. The cross sections of the spreading elements 13 are oriented in such a way that they are oriented in the direction of the tissue of the urethra 18. By means of this specific alignment of the triangles, a particularly high pressure can be exerted on the tissue, leading to particularly efficient treatment.

Furthermore, it can be seen from the exemplary embodiment illustrated in FIG. 4 that the spreading elements 13 have a cavity 23. This cavity 23 can extend over the entire length of the spreading elements 13 or can be limited to specific regions. These cavities 23 can serve to receive medicaments, which are then delivered directly in the urethra 18. Once the biodegradable material dissolves, the medicament is delivered to the body in a predetermined manner. In this case, it can be determined, in particular by the choice of the wall thickness of the spreading element 13, how quickly or at what rate the medicament is delivered. Similarly, it is conceivable for the spreading elements 13 to have further openings or pores through which the medicament is delivered to the body.

As the implant 10 is introduced into the urethra 18, the spreading elements 13 rest directly against the bracing means 16. The angle between the bracing elements 14, 15 and the bracing means 16 is minimal in this phase. In this state, the implant 10 is introduced into the urethra 18 with the distal end 17 in front. As soon as the distal end 17 is projecting into the bladder neck 19, a force is exerted on the proximal end 20 or the bracing means 16 in the direction of the arrow 24, i.e. in the proximal direction. This can be done by means of a thread, a rod or forceps (not illustrated). As a result of this application of force, the pressure means or the parallelograms open out and move in the direction of arrow 25 against the tissue of the urethra 18. During this opening of the implant 10, the diameter of the implant 10 increases, in particular at the distal end 17, with the result that the extensions 22 of the distal bracing elements 15 project beyond the diameter of the urethra 18 and act as a kind of anchor. The implant 10 is prevented from slipping back into the urethra 18 by these extensions 22. The implant 10 is opened out on an individual basis, depending on the urethra 18 to be treated or depending on the anatomy of the patient. As a result of the latching effect of the joints 21, the diameter of the implant 10 can be enlarged and fixed at least approximately continuously or in stages. FIG. 2 illustrates the implant 10 in an end position. Here, the spreading elements 13 press against the tissue of the urethra 18, while the extensions 22 rest on the bladder neck 19 and prevent the implant 10 from slipping in the proximal direction 24.

After completion of the treatment, the implant 10 dissolves automatically or can be pulled back out of the urethra 18. For this purpose, a tensile force can again be exerted on the bracing means 16 in the direction of arrow 24. An increased tensile force causes the joints 21 to break, with the result that the bracing elements 14, 15 move beyond the maximum position and the parallelograms fold up in the opposite direction than before (FIG. 3). As a result, the diameter of the implant 10 is reduced and it can be easily pulled out of the urethra 18. For this purpose, it is possible once again to use a thread, a rod or forceps, for example.

### LIST OF REFERENCE SIGNS

10 implant
11 pressure means structure
12 pressure means
13 spreading element
14 proximal bracing element
15 distal bracing element
16 bracing means
17 distal end
18 urethra
19 bladder neck
20 proximal end
21 joint
22 extension
23 cavity
24 direction of arrow
25 direction of arrow

1-19. (canceled)

20. An implant for expanding a urethra of a person by applying a local ischemic pressure to the tissue of the urethra using a pressure means structure, which has at least two pressure means, wherein the implant can be introduced into the urethra with a distal end in front, wherein the pressure means each have a spreading element and two bracing elements, namely a proximal and a distal bracing element, wherein the bracing elements are each articulated movably at a first end to the spreading element and movably at a second end to a central bracing means.

21. The implant as claimed in claim 20, wherein the two ends of the bracing elements each have a joint, by means of which they are connected movably to the respective spreading element and to the central bracing means.

22. The implant as claimed in claim 20, wherein the pressure means structure is made up of three pressure means, wherein the three pairs of bracing elements are movably connected by their second ends to the common central bracing means and to a respective spreading element.

23. The implant as claimed in claim 22, wherein the three spreading elements of the three pressure means are arranged at an angular spacing of 120° with respect to one another around the central bracing means.

24. The implant as claimed in claim 20, wherein the spreading elements of a plurality of pressure means are aligned parallel to one another.

25. The implant as claimed in claim 20, wherein the two bracing elements of a pressure means are aligned parallel to one another and are movable parallel to one another.

26. The implant as claimed in claim 20, wherein the pressure means can be brought into a folded state, wherein the bracing elements and the spreading elements can be moved in the direction of the bracing means, thereby minimizing the distance between the bracing elements, the spreading elements and the bracing means.

27. The implant as claimed in claim 20, wherein the pressure means can be brought into an opened state, wherein the bracing elements and the spreading elements can be moved away from the bracing means, thereby maximizing the distance between the spreading elements and the bracing means, and the spreading elements thus open up the urethra.

28. The implant as claimed in claim 20, wherein the bracing means has a distal and a proximal end, wherein it can be pushed into the urethra with the distal end in front, and wherein the pressure means structure can be opened with the pressure means by the action of force on the proximal end in the proximal direction.

29. The implant as claimed in claim 20, wherein the distal bracing elements each have an extension which projects beyond the first end, wherein, in the opened state, the pressure means can be fixed with the extensions in a bladder neck of the urethra.

30. The implant as claimed in claim 29, wherein the extension has a curvature.

31. The implant as claimed in claim 20, wherein the joints between the spreading elements and the bracing elements and between the bracing elements and the bracing means can be rotated only over a limited angular range (angle limitation).

32. The implant as claimed in claim 20, wherein the joints have a latch between the spreading elements and the bracing elements and between the bracing elements and the bracing means, thus enabling them to be locked in a selectable relative angular position.

33. The implant as claimed in claim 31, wherein the angular limitation of the joints can be lifted by the application of force to the bracing means in the proximal direction, and the pressure means can be brought back into a folded state via the opened state in order to pull the implant out of the urethra.

34. The implant as claimed in claim 20, wherein the spreading elements are of hollow design for the metered reception of a substance.

35. The implant as claimed in claim 20, wherein the spreading elements have a polygonal, a triangular or drop-like cross section, wherein the spreading elements are oriented in such a way that they are aligned with a pointed portion of the cross section in the direction of the tissue of the urethra to be treated.

36. The implant as claimed in claim 20, wherein the spreading elements and/or the bracing means and/or the bracing elements and/or the joints are produced from a biodegradable material.

37. The implant as claimed in claim 36, wherein the spreading elements, the bracing means and the bracing elements have different thicknesses or wall thicknesses, as a result of which the spreading elements, the bracing means and the bracing elements degrade at different rates.

38. The implant as claimed in claim 35, wherein the pressure means structure has further elements for fixing within the urethra, wherein these elements are designed in such a way that they degrade first.
